(19) United States
(12) Patent Application Publication
(10) Pub. No.: US 2023/0335294 A1
(43) Pub. Date: Oct. 19, 2023

(54) SYSTEM AND METHOD FOR DETECTING AND MONITORING MEDICAL CONDITION AND IMPAIRMENT

(71) Applicant: NeoGeminae Inc., Coral Gables, FL (US)

(72) Inventor: Mark Wolff, Cary, NC (US)

(21) Appl. No.: 18/136,842

(22) Filed: Apr. 19, 2023

Related U.S. Application Data
(60) Provisional application No. 63/332,379, filed on Apr. 19, 2022.
(60) Provisional application No. 63/332,367, filed on Apr. 19, 2022.

(51) Int. Cl. G16H 50/50; A61B 5/11; A61B 5/00; G06V 40/20

(52) U.S. Cl. CPC G16H 50/50; A61B 5/1104; A61B 5/6823; G06V 40/23

(57) ABSTRACT

A system and method for detection and monitoring of medical conditions and physical and/or cognitive impairments are provided. In embodiments, the system of the present invention utilizes inertial measurements and, in some embodiments, imaging to quantify body movements and conditions. In embodiments, the present system incorporates one or more inertial measurement unit (IMU) sensors, a camera, computer vision, and a series of standardized body movements. In additional embodiments, the present system incorporates one or more IMU sensors and a series of standardized body movements without the use of a camera or computer vision. In embodiments, the present system creates and stores a personalized “digital twin” of a patient or subject's functional musculoskeletal capabilities. Such digital twin acts as a reference model of the patient or subject.

### CROSS-REFERENCE TO RELATED APPLICATIONS

This application claims priority pursuant to 35 U.S.C. 119(e) to co-pending U.S. Provisional Patent Application Ser. No. 63/332,367, filed Apr. 19, 2022, the entire disclosure of which is incorporated herein by reference. The application also claims priority pursuant to 35 U.S.C. 119(e) to co-pending U.S. Provisional Patent Application Ser. No. 63/332,379, filed Apr. 19, 2022, the entire disclosure of which is incorporated herein by reference.

### FIELD OF THE INVENTION

The present invention relates generally to medical condition and impairment detection, monitoring, and treatment. More specifically, the present invention is concerned with a system and method for monitoring and detecting physical conditions, impairments, and effectiveness of treatment by measuring acceleration, rotation, and linear and angular translation during standardized movements, quantifying baseline measurements, and measuring and/or monitoring differential from such baseline conditions.

### BACKGROUND OF THE INVENTION

Historically, medical diagnoses and treatments have generally followed a one size fits all or one size fits most approach relating to many medical conditions. Nevertheless, individual patients are often affected differently by medical conditions and/or react differently to treatments when compared to other patients. In some pain and discomfort management scenarios, medical professionals adjust prescribed treatments and/or dosages based on an assessment of a patient's current pain or discomfort, however such assessment is typically made largely or entirely from the patient's answers to questions requiring the patient to arbitrarily rate how the patient feels, often on a numbered scale. It can be difficult for medical professionals to determine whether one patient's use of a number scale is equivalent to another patient's number scale or how number scales differ from patient to patient. Thus, such methods incorporating patients rating their own pain and discomfort are largely ineffective for monitoring and assessing medical conditions and the effectiveness of treatments. Accordingly, it would be beneficial to have a system capable of quantifying a patient's condition and response to treatment to aid in a more effective, individualized treatment plan.

In recent years, value-based healthcare and “patient centricity” have emerged as global models for how healthcare is delivered and paid for, aiming to drive costs down and improve patient outcomes. The overall goal is to incentivize care providers to better engage patients and to adopt evidence-based medicine in diagnosis and treatment decisions more consistently. The emergence of digital health devices and the Internet of Medical Things (IoMT) have been driving forces for this transformation in healthcare by providing more connectivity between patients, clinicians, machines, and care environments. Digital health and the IoMT are critical in addressing many challenges and opportunities related to healthcare delivery inefficiencies, access, costs, quality, and personalization.

Substantial research has been conducted on the application of sensing technology to study and understand aspects of human motion. Clinicians and researchers have previously used sensors to compare movement in normal and pathological states in efforts to quantify therapeutic effects. Nevertheless, such analyses can be complex, expensive, qualitative, and inconsistent. Accordingly, it would be beneficial to have a system which more effectively and efficiently utilizes sensor technologies in quantifying human motion.

Recent advances have been made in the development of small, inexpensive, sensors, such as but not limited to multi-axis inertial measurement units (IMUs) and highly sensitive and accurate image analysis systems. Such devices can accurately sense rotation and acceleration via gyroscopes, accelerometers, and magnetometers and can measure discrete movement via computer vision and three-dimensional (3D) imaging with infrared and/or LIDAR sensing. It would be beneficial to have a system and method configured to incorporate multi-axis IMUs and image analysis systems into medical condition and impairment detection, monitoring, and analysis.

Current work in the area of “pain quantification” focuses on bioelectric techniques of measurement such as electroencephalography (EEG), which is a technique used to track electrical activity of the brain through the placement of electrodes on the scalp, and electromyography (EMG), which is a technique that allows for the recording of electrical impulses that are generated by muscle activity. These analyses are either made passively or via stimulation (evoked response). Nevertheless, they are complex, difficult to measure outside of highly controlled environments, and often produce noisy or inconsistent signals. As a result, bioelectric measurements are difficult to reliably interpret for pain management. Accordingly, it would be beneficial to have a system and method for quantification of pain with easy, reliable measurements.

Moreover, presently there is not a portable, non-invasive system for objective and quantifiable assessment of intoxication by neuroactive agents, such as but not limited to cannabis intoxication, which is similar to a breathalyzer for alcohol intoxication. Intoxication by neuroactive agents, often substances of abuse, is the result of well understood biochemical and pharmacological processes. Pharmacologically active substances, particularly substances that affect the nervous system do so by acting at specific components of the nervous system. Such effect of these substances often results in behaviors that are well understood scientifically. So, while intoxication can be quantified through the testing of blood and other tissue, it is also possible to define intoxication from idiotypic behavior of an intoxicated individual by assessing behavior at the neuromuscular level.

Evaluating balance, coordination, attention, lethargy, hyper-activity tremors, and many more idiotypic behaviors can be used to detect intoxication, however such assessments are not routinely applied for intoxication detection, as they have been exceptionally difficult to measure and quantify. Accordingly, invasive blood or tissue born testing has been relied on heavily. It would be beneficial to have a system and method for immediate assessment of neuroactive agent intoxication, such as cannabis or alternative drug intoxication, without requiring blood testing.

The present state of the art in human motion analysis and impairment detection does not combine the physics of human motion (acceleration and rotation) with the discrete measurement of that motion as a function of translation of the body through space and time (computer vision). Heretofore, there has not been a system and method for medical condition and impairment detection and monitoring with the advantages and features of the present invention.

### SUMMARY OF THE INVENTION

The present invention comprises a system and method for individualized medical condition and physical and/or cognitive impairment detection and monitoring. In exemplary embodiments, the system of the present invention utilizes inertial measurements and, in some embodiments, imaging to quantify body movements and conditions. In embodiments, the present system incorporates one or more inertial measurement unit (IMU) sensors, a camera, computer vision, and a series of standardized and ad hoc body movements. In additional embodiments, the present system incorporates one or more IMU sensors and a series of standardized body movements without the use of a camera or computer vision.

In an exemplary embodiment, the present invention further utilizes machine learning techniques applied to data collected from sensor analysis and, if applicable, image analysis of patients or subjects. This data is configured for use in studying clinically relevant movements and in identifying correlates between pathology, impairment, pain, discomfort, and disequilibrium. Such analyses facilitate discrete quantification of therapeutic effects and outcomes of pharmacologic and mechanical therapies in clinical settings as well as physical and cognitive impairments, including but not limited to intoxication by neuroactive agents.

Moreover, the present system is capable of being deployed outside of a clinic for applications in remote (e.g., in-home) patient monitoring, both for post-acute medical conditions and chronic medical conditions requiring long-term monitoring. This improves many clinical diagnostics protocols, including but not limited to for musculoskeletal, orthopedic, and neurological conditions, by reducing the time, cost, and subjectivity of manual assessments.

In further embodiments, the present system is capable of being deployed for applications in remote, non-invasive impairment detection (e.g., by law enforcement in the field). This improves impairment detection protocols, particularly for but not limited to detection of impairment by neuroactive agents other than alcoholic substances. Nevertheless, in additional embodiments, the present system is utilized for detection of impairment by alcohol.

The present invention combines the physics of human motion (i.e., acceleration and rotation) with discrete measurement of that motion as a function of translation of the body through space and time. The system further integrates machine learning techniques to assist in accurate and consistent measurements with a high degree of resolution to better assess a subject's ability to control movements using simple and easy to use hardware.

In embodiments, a combination of physical data from an accelerometer and a gyroscope, image data from computer vision component(s), and synchronized time data accommodates use of analytic models and algorithms to provide a high level of resolution, consistency, and applicability with relatively simple hardware compared to other methods of human motion analysis. Such combination of these three components provides the ability of the present system to perform rapid, accurate, and quantifiable assessments of the human musculoskeletal system.

In another embodiment of the present invention, an IMU sensor includes an integrated microprocessor configured for processing acceleration and rotation data itself and is utilized with the present system without a camera and computer vision. In this embodiment, the IMU is a self-contained measurement and assessment device.

In an embodiment, upon discrete measurements of human motion with the application of IMU sensor(s) and optional computer vision, the present system creates and stores a personalized “digital twin” of a patient or subject's functional musculoskeletal capabilities. Such digital twin acts as a reference model of the patient or subject from which factors such as disease progression; therapeutic intervention, whether surgical, mechanical, or pharmacological; balance; coordination; attention; lethargy; hyper-activity tremors; and more idiotypic behaviors can reliably, consistently, and objectively be assessed, reviewed, and compared through quantitative analysis. Digital twins of the present invention can be used for comparison to a patient or subject's baseline and/or a global nominal state model.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof

### BRIEF DESCRIPTION OF THE DRAWINGS

Embodiments of the present invention are described in detail below with reference to the attached drawing figures, wherein:

FIG. 1 is a schematic drawing of a medical condition and impairment detection and monitoring system and method embodying the present invention.

FIG. 2 is a schematic drawing showing system architecture of a medical condition and impairment detection and monitoring system of the present invention.

FIG. 3 shows a front, perspective view of an embodiment of the present invention including a rolling cart mounting components of a medical condition and impairment detection and monitoring system.

FIG. 4 is a dose-response curve illustrating applicability of the medical condition and impairment detection and monitoring system and method of the present invention.

FIG. 5 shows an embodiment of a human key point identification scheme for use in association with a camera and computer vision of the present invention. Each numbered key point in FIG. 5 represents an (x,y) coordinate in space at a certain time.

FIG. 6 is an elevational representation of an embodiment of camera and subject positioning of the present invention, showing positions of the subject for computer vision in the frontal plane and the sagittal plane.

FIG. 7 is a back, upper, perspective view of inertial measurement unit (IMU) placement on a subject embodying the present invention.

The drawing figures do not limit the present invention to the specific embodiments disclosed and described herein. The drawings are not necessarily to scale, emphasis instead being placed upon illustrating the principles of the invention.

### DETAILED DESCRIPTION OF THE PREFERRED EMBODIMENTS

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The present invention comprises a system and method of quantifying bodily motion for diagnostic and treatment purposes. In an exemplary embodiment, the present system comprises a system and method of quantifying bodily motion to detect and/or monitor medical condition(s), impairment(s), and/or treatment(s). In exemplary embodiments, the system of the present invention utilizes inertial measurements and, in some embodiments, imaging to quantify body movements and conditions. In embodiments, the present system incorporates one or more inertial measurement unit (IMU) sensors, a camera, computer vision, and a series of standardized and ad hoc body movements. In additional embodiments, the present system incorporates one or more IMU sensors and a series of standardized body movements without the use of a camera or computer vision.

In an exemplary embodiment, the system includes a computing device having a processor configured to process data from the sensors and optional camera and a memory configured to store results. In a preferred embodiment, the system further includes a user interface configured to allow a user to control operation of the sensor(s) and optional camera and to display the data and results.

In an exemplary embodiment of the present invention, the system is configured to objectively measure a subject's pain by quantifying motion in terms of acceleration, rotation, and measurement of the linear and angular translation of key points of interest on the subject's body via computer vision. In embodiments, this system is used in health and medicine industries to quantify human motion and measure pain. In further alternative embodiments, the present system is configured for use in the animal health and veterinary medicine space to quantify motion of other types of animals to measure pain.

In another exemplary embodiment, the system is configured to objectively determine whether a subject is impaired by quantifying motion in terms of acceleration, rotation, and measurement of the linear and angular translation of key points of interest on the subject's body. In embodiments, this system is used efficiently and non-invasively to identify physical and/or cognitive impairment.

In an exemplary embodiment, the present invention utilizes technical and mathematical integration of telemetry from one or more inertial measurement units (IMUs) and region of interest (ROI) identification and tracking. In some embodiments, the present system further utilizes a digital camera, computer vision, and human pose estimation techniques for ROI identification and tracking.

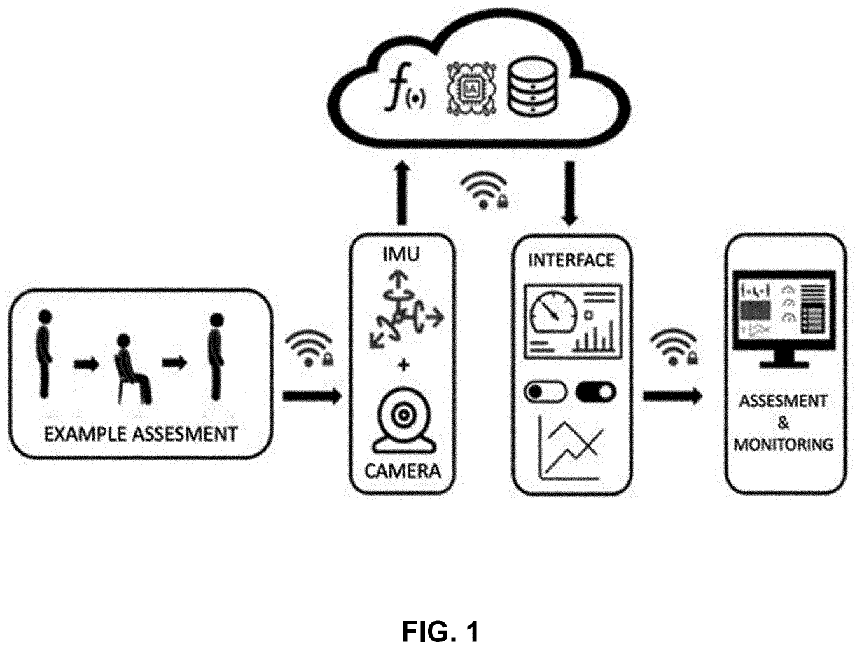

Referring to the drawings in more detail, FIG. 1 shows a schematic drawing of a system and method for detecting and monitoring medical conditions and impairments embodying the present invention. In the embodiment shown in FIG. 1, the system includes an IMU and a camera in communication with a system computing device having a microprocessor configured for assessment and monitoring based on input from the IMU sensor(s) and camera. In this embodiment, the system further includes a user interface accessible via a display on either on the system computing device or another connected computing device, such as a mobile device or computer. In this embodiment, a patient or subject performs a series of physical movements with the IMU configured for measuring the acceleration and rotation of said movements and the camera positioned to record said movements. In this embodiment, the system computing device is programmed with computer vision capabilities for identifying key points of interest on the patient or subject's body and tracking linear and angular translation of those key points over time during the physical movements. The system computing device is configured to further process the IMU and computer vision data to create a digital representation of the patient or subject's movements. In an exemplary embodiment, information regarding the digital representation of the patient or subject's movements is displayed on the user interface. In the embodiment shown in FIG. 1, the system further comprises a database with memory for storing historical assessment data. In an embodiment, the database memory comprises non-transitory storage media. In an embodiment, the database is a cloud-based database. In an exemplary embodiment, the system components are connected via a communications network, such as but not limited to the internet or an intranet. In embodiments, said connections are wireless or wired.

In another embodiment of the present invention, an IMU sensor includes an integrated microprocessor configured for processing acceleration and rotation data itself and is utilized with the present system without a camera and computer vision. In this embodiment, the IMU is a self-contained measurement and assessment device. Such devices are commonly referred to as edge devices as part of the Internet of Things (IoT). In an exemplary embodiment, the IMU further includes an indicator light, such as but not limited to an LED light. In embodiments, such indicator light can be configured to light up in one or more colors depending on the assessment being conducted.

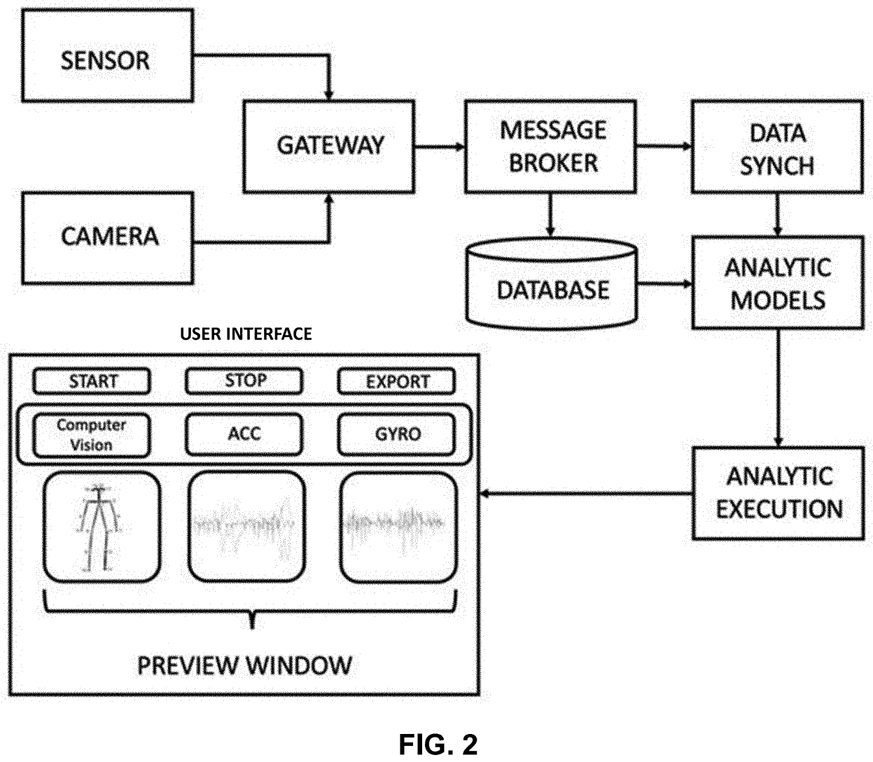

FIG. 2 is a schematic drawing showing system architecture of an exemplary embodiment of a medical condition and impairment detection and monitoring system and method of the present invention. In this embodiment, the system includes a sensor and a camera which each provide inputs to a gateway which communicates the inputs to a message broker module configured for communication with a system database and a data synchronization module. The system further utilizes programmed analytic models to perform analytic execution of the sensor and camera inputs to create an output of a digital representation of movements recorded by the sensor and camera. In an exemplary embodiment, the digital output is viewable via a user interface. In an exemplary embodiment, the user information displays information regarding a patient or subject's movements, including computer vision, accelerometer, and gyroscope information. In an exemplary embodiment, the user interface is configured for displaying measurements synchronized with time. In an exemplary embodiment, the user interface is configured with a series of functions, including but not limited to start, stop, and export data.

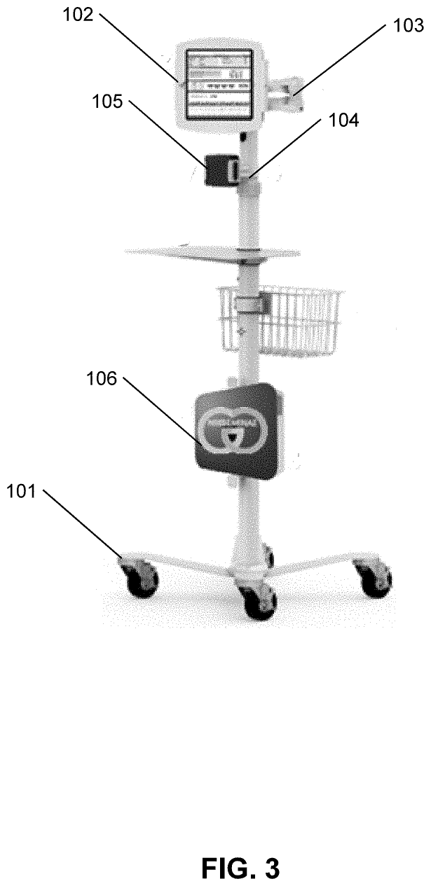

FIG. 3 shows an exemplary embodiment of a medical condition detection and monitoring system of the present invention including a rolling stand mounting system components. In this embodiment, the system includes a rolling stand with wheels 101, such as but not limited to caster wheels. In a preferred embodiment, the wheels comprise locking wheels. In this embodiment, the system includes a display 102 configured for displaying a system user interface. In embodiments, the display comprises a mobile device, such as a tablet with a display, or a monitor. In an exemplary embodiment shown in FIG. 3, the display 102 is connected to an adjustable articulating arm 103 for adjustment as desired by a user. In this embodiment, the system includes an adjustable camera holder 104 configured for adjustably mounting a camera 105. In alternative embodiments, a camera integrated into the display is utilized, such as an internal tablet camera or monitor camera, instead of an external camera. In further embodiments, both an integrated display camera and an external camera are utilized in association with computer vision. In the embodiment shown in FIG. 3, the system further comprises a portable computing device and sensor gateway 106. In embodiments, the portable computing device may be integrated with the display. In the embodiment shown in FIG. 3, the rolling stand further comprises a work surface and storage space. In embodiments, storage space is utilized to house IMU sensor(s) and attachment stickers or tape. In embodiments, the system is further equipped with a rechargeable battery pack to power the system components. In alternative embodiments, the system includes wired connection to a power source, such as an electrical outlet plug.

In an exemplary embodiment, the present system is configured to process and technically and analytically synchronize IMU sensor data and computer vision data, in association with time series data, so that such data can be combined into a single data set. The combined and synchronized data of the present invention provides the ability to apply new analyses and generate algorithms for application in clinical and/or bodily impairment assessment and monitoring. Thus, combining a time signature; the measurement of forces and rotational motion, which describe how the body moves in physics-based values; and computer vision, which identifies key points on the body and tracks those points through space and time as a series of coordinates (x,y), allows for values such as speed, range, momentum, acceleration, rotation, velocity, distance, displacement, work, energy, and other related parameters to be derived.

In an embodiment, upon discrete measurements of human motion with the application of IMU sensor(s) and optional computer vision, the present system creates and stores a personalized “digital twin” of a patient or subject's functional musculoskeletal capabilities. Such digital twin acts as a reference model of the patient or subject from which factors such as disease progression; therapeutic intervention, whether surgical, mechanical, or pharmacological; balance; coordination; attention; lethargy; hyper-activity tremors; and more idiotypic behaviors can reliably, consistently, and objectively be assessed, reviewed, and compared through quantitative analysis. In an embodiment, the digital twin is a digital representation of the patient or subject modeled from real world data, such as but not limited to data from electronic health records (EHRs), disease registries, historical data, wearable sensors, and computer vision, in real time or near real time. Such digital twins can be extensible and continually improved by the introduction of additional and complementary data measured from the patient or subject. The digital twin represents actual measured data rather than being a simulation or imitation over time. This approach improves outcome assessment and therapeutic effectiveness with a particular focus on personalized diagnosis and therapy. In exemplary embodiments, a patient or subject has a baseline digital twin, which represents that patient or subject's normal or baseline condition, and an active digital twin, which represents that patient or subject's current condition.

In an exemplary embodiment of the present invention, IMU(s) utilized are integrated 9-axis absolute orientation sensors combining a 3-axis solid-state acceleration sensor, a 3-axis gyroscope, and a 3-axis geomagnetic sensor capturing data at 30 to 100 Hz. In an exemplary embodiment, a utilized digital camera has a minimum resolution of 480×480 with a minimum capture rate of 15 frames per second (FPS). In an exemplary embodiment, data from an IMU are collected at a specific frequency (30 to 100 Hz) across six parameters, three from the accelerometer in units of gravitational force (g) and three from the gyroscope in units of degrees per second (deg/sec), as a function of time in seconds. Accordingly, the data collected includes x-axis, y-axis, and z-axis data in units of gravitational force (g); x-axis, y-axis, and z-axis data in degrees per second (deg/s); and time data in seconds. In alternative embodiments, alternative types of IMUS are utilized.

In other embodiments, IMU sensor data is captured at 1 to 1,000 Hz, 10 to 500 Hz, 15 to 300 Hz, 20 to 200 Hz, 20 to 100 Hz, 40 to 100 Hz, 50 to 100 Hz, approximately 30 Hz, approximately 35 Hz, approximately 40 Hz, approximately 45 Hz, approximately 50 Hz, approximately 55 Hz, approximately 60 Hz, approximately 65 Hz, approximately 70 Hz, approximately 75 Hz, approximately 80 Hz, approximately 85 Hz, approximately 90 Hz, approximately 95 Hz, approximately 100 Hz, about 30 Hz, about 35 Hz, about 40 Hz, about 45 Hz, about 50 Hz, about 55 Hz, about 60 Hz, about 65 Hz, about 70 Hz, about 75 Hz, about 80 Hz, about 85 Hz, about 90 Hz, about 95 Hz, about 100 Hz, or other data output ranges. In embodiments, the IMU(s) of the present invention are configured to transmit data via Wi-Fi, Bluetooth, alternative radio frequency, other cellular connection, or an alternative transmission method over a communications network.

In exemplary embodiments utilizing a camera and computer vision, the digital camera has a minimum resolution of 480×480 with a minimum capture rate of 15 frames per second (FPS). In other embodiments, the digital camera has a minimum resolution of 320×200, 320×240, 640×480, 720×480, 800×600, 1024×768, 1280×1024, 1600×1200, 1280×720, 1440×1080, 1920×1080, or other minimum resolution.

In embodiments, a digital camera utilized with the present system has a minimum image capture rate of 5 FPS, 6 FPS, 7 FPS, 8 FPS, 9 FPS, 10 FPS, 11 FPS, 12, FPS, 13 FPS, 14 FPS, 16 FPS, 17 FPS, 18 FPS, 19 FPS, 20 FPS, 21 FPS, 22 FPS, 23 FPS, 24 FPS, 25 FPS, 30 FPS, 35 FPS, 40 FPS, approximately 10 FPS, approximately 15 FPS, approximately 20 FPS, approximately 25 FPS, approximately 30 FPS, approximately 35 FPS, approximately 40 FPS, about 10 FPS, about 15 FPS, about 20 FPS, about 25 FPS, about 30 FPS, about 35 FPS, about 40 FPS, or other minimum capture rates.

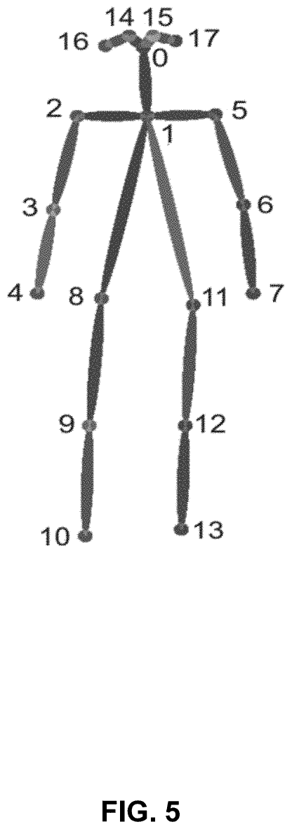

In an exemplary embodiment of the present invention, data from a camera are collected at a specific frame rate and saved as coordinates of key points, identified via human pose estimation. Key points are defined as specific areas associated with an anatomical landmark, for example a subject's right shoulder or left knee. While recording with the present system, each key point position is recorded as a coordinate (x,y) at a point in time. FIG. 5 shows a non-limiting example of a key point identification scheme of an exemplary embodiment of the present invention. The key point identification scheme in the embodiment shown in FIG. 5 includes 18 key points each associated with an anatomical landmark. In an exemplary embodiment, the system processor is configured to identify desired key points on a patient or subject's body from video input from a system camera.

The frequency of data point collection from the IMU sensor and optional camera of the present invention is synchronized such that acceleration, rotation, and key point coordinate position in time and space are aligned for a given point in time and over the time frame of the assessment. In a preferred embodiment, the present system is configured to automatically conduct such synchronization, allowing a user to seamlessly use the combined data for assessment.

The sensors of the present invention are configured to make highly accurate measurements of a subject's movement as related to established assessment protocols for musculoskeletal and neurological disorders and/or physical and cognitive impairment (i.e., measurements of forces due to acceleration, rotation, and angular and linear body position). In an exemplary embodiment, data from the sensor(s) are processed for objective and quantitative assessment of a subject's pain, discomfort, and/or disequilibrium. Such assessment is then communicated to a user to support clinical decision making and to quantify clinical outcomes associated with therapeutic intervention. In another exemplary embodiment, data from the sensor(s) are processed for objective and quantitative assessment of whether a subject is impaired. Such assessment is then communicated to a user of the system, such as law enforcement or a medical professional.

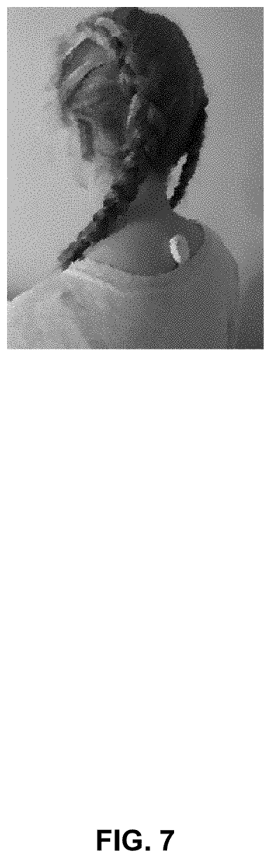

In an exemplary embodiment, an IMU is placed on the subject's body via a two-sided adhesive strip. Positioning of the IMU sensor is dependent on the specific assessment to be carried out. In alternative embodiments, an IMU is attached to the subject's body via other means, such as but not limited to via strap(s), peel and stick adhesive, or a clip configured for attachment to the subject's clothing. In an exemplary embodiment of the present invention, an IMU is positioned on the seventh cervical (C7) vertebrae of a subject, as shown in FIG. 7.

In alternative embodiments, an IMU is positioned on a subject's sixth cervical (C6) vertebrae, fifth cervical (C5) vertebrae, first thoracic (T1) vertebrae, second thoracic (T2) vertebrae, third thoracic (T3) vertebrae, fourth thoracic (T4) vertebrae, fifth thoracic (T5) vertebrae, sixth thoracic (T6) vertebrae, sternum, right shoulder, left shoulder, right hip, left hip, right knee, left knee, right ankle, left ankle, right elbow, left elbow, right wrist, left wrist, forehead, or another anatomical location.

In embodiments further utilizing a camera and computer vision, once the IMU sensor is properly placed, the subject is positioned in front of a camera. In an exemplary embodiment, the camera is positioned such that the subject's entire body is visible in the frame either in the frontal or sagittal plane depending on the particular assessment being made.

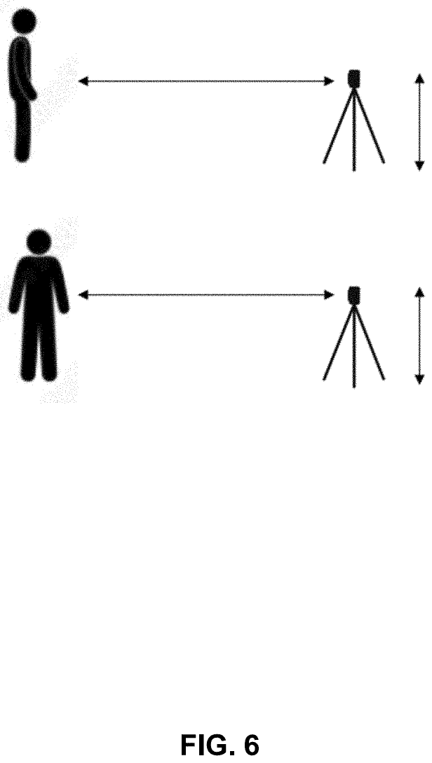

In an embodiment, with the IMU in proper placement on the subject, the camera of the present system is configured to record the subject from a frontal view, with the subject facing the camera; from a side or lateral view, with the with the camera positioned a distance off the ground; or both. In an exemplary embodiment, the camera is positioned approximately two to three meters away from the subject and approximately a meter off the ground. An embodiment of camera and subject positioning is shown in FIG. 6. Alternatively, a camera can be positioned a shorter or further distance away from the subject, higher or lower from the ground, or not utilized at all without diverting from the present invention.

In an exemplary embodiment, the subject performs a standard set of functional motions and/or movements for testing mobility and balance while the present system records relevant data. As a non-limiting example, the subject performs a 30-second chair stand test, a time up and go test, and/or a 4-stage balance test, such tests as defined by the U.S. Centers for Disease Control and Prevention. In another exemplary embodiment, a subject is instructed to perform a standard set of motions and/or movements while the system records relevant data. In embodiments, the standard motions and/or movements performed differ depending on the condition, impairment, or treatment being tested.

In an exemplary embodiment, data is collected using the present invention, resulting in a table or matrix of time series data describing values from the accelerometer, gyroscope, and key point coordinates from the camera. Such data allow for a very detailed and relevant model or digital twin of the human musculoskeletal system and its control by the nervous system, which in turn facilitates effective personalized and precise medicine and/or impairment assessments. Moreover, with the present invention, human motion can be measured and described in terms of numerous physics-based quantities, such as scalar, vector, and derived measures, including but not limited to acceleration, rotation, speed, velocity, distance, displacement, momentum, work, and energy.

The present invention accommodates complete digitization and automation of collection and assessment of a patient or subject performing one or more mobility tests. The system results in much more reproducible and accurate measures for each patient or subject, which ultimately makes possible discrete measurement changes in a patient over time based on multiple assessments and measurements of impairment. Additionally, the patient's motion can be described in much greater detail by the present system because of values derived which could not previously be measured.

For example, the 30-second chair stand test measures how many times an individual can sit and stand in 30 seconds. The present system can be used to automate this assessment and also to drive metrics beyond the number of sit to stand iterations. The consistency of each iteration, the reproducibility of each iteration, and the rate at which each interval is achieved, that is whether the subject is slowing down or speeding up, can be calculated. Further, the stability of each iteration in terms of the variability of the motion of sitting and standing can be measured. The system can also be used to measure various factors associated with speed, work, and energy related to each motion. Each of these recorded measures can be combined to paint a detailed digital picture of the subject. Such a picture can then be used to compare results over time and to better pinpoint where or how a patient is or is not improving given a particular therapy.

Integrated telemetry from the aforementioned three sensing modalities and algorithmic analysis of those data create a quantitative and objective assessment of the subject relative to the amount of pain, discomfort, and/or disequilibrium the subject is experiencing due to a musculoskeletal or nervous system pathology. Over time, in an embodiment of the present invention, the system is utilized to help therapeutically and/or pharmacologically calibrate treatment based on measured movement as recorded and processed by the three sensing data streams.

The present invention is capable of aiding in therapeutic and pharmacological calibration largely because of the reflexive and instinctive nature of the body's response to musculoskeletal pain. For example, people limp to avoid putting extra pressure on a sprained ankle or other injury. The present invention allows such reflexive behavior (i.e., the limp) to be measured and modeled mathematically from the sensor data. The same assessment can then be continually performed after the subject is dosed with a specific amount of analgesic or other treatment, and absolute change and percent change of the data collected can be tracked.

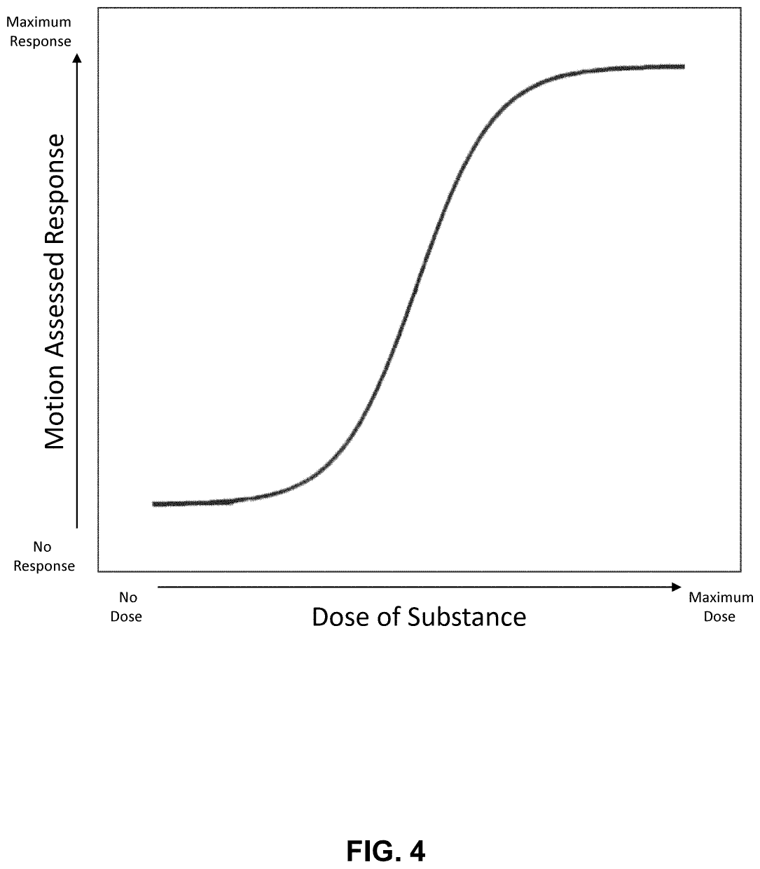

Pre- and post-treatment motion is measured, and the dose of an analgesic can be calibrated based on such data. This method can effectively replicate a “dose response curve” between the amount of pain medication and the change in motion from a subject's “pain state” to the subject's “nominal state.” Such dose-response or exposure-response relationship defines the magnitude of the response of an organism as a function of exposure to (i.e., doses of) a stimulus or stressor (typically but not limited to a chemical) after a certain exposure time. An example of such a dose response curve is shown graphically in FIG. 4.

Embodiments of the present invention utilize mathematical representation of a digital twin of the subject to be used as a comparator and reference point for motion quantification. In an exemplary embodiment of the present invention, a subject's motion data are collected in a nominal or healthy state to establish a baseline model for use as a comparator for follow-up assessments. In a preferred embodiment, use of the present invention includes regular assessment to update baseline measurements. In embodiments, regular assessment is used to track a condition and therapeutic effect over time.

In further embodiments, motion data are collected following an injury or the diagnosis of a relevant pathology. In a preferred embodiment, these data are used to create a model and baseline comparator in an injured or diseased state. Furthermore, these data can be used during follow-up assessments after a therapeutic intervention for comparison and for measurement of whether there was a quantifiable change away from an injured state and toward a nominal state. From this analysis, an objective and quantifiable value is created that first relates to the level of efficacy of a treatment in relieving pain and then the body's reflexive reaction to the treatment.

In embodiments of the present invention, a patient or subject can be assessed using the present system at any time, and the results of the assessment can be compared to a global nominal state model. Such comparisons allow for quantitative comparisons between different subjects, such as but not limited to quantitative comparisons between different subjects having the same or similar medical diagnoses. For example, if two patients with similar demographic and pathology profiles have the same surgery but have different outcomes after surgery, the present system can be utilized to quantify such difference. For instance, the present system is capable of measuring that a first patient is at 85% of nominal or “normal” value at six weeks post-operation, whereas a second patient is at 50% of nominal value at six weeks post-operation. In this scenario, utilizing data measured with the present invention, it can be accurately determined that the first patient has a 35% better outcome in terms of a specific mobility assessment than the second patient at six weeks post-operation.

In an exemplary embodiment, a subject's motion data are collected in a nominal or healthy state to establish a baseline model for use as a comparator for later assessments. In embodiments, such nominal or healthy state measurements are taken as part of a driving test or driver's license renewal, similar to a driver eye examination, or as part of routine medical checkup procedures, similar to measuring height and weight. In further embodiments, such measurements are taken as part of the intake process when someone is arrested, similar to the process of taking fingerprints.

In an embodiment, a global nominal state model is continually updated with additional data to refine its accuracy. In an embodiment, the system processor is configured to be adaptive and capable of learning based on the aggregation and segmentation of individual data. Such adaptive models allow the system to better segment patient populations relative to their disease state and their outcomes potential.

The present invention is further capable of differentiating, via mathematical modeling of the present system, between an instinctive, reflexive, neuronally-mediated motion and that of a “fake” limp. Such an assessment is capable of use as a tool when making decisions related to prescription of narcotics and addictive analgesic substances, helping to mitigate addiction and abuse of such substances. Thus, the present invention is capable of being used as a lie detector of sorts for pain.

In an example of a use of the present invention, after a subject sprains an ankle, an assessment is conducted using the present system to provide values relating to the subject's ability to perform specific motions (e.g., standing from a sitting position; sitting from a standing position; turning around 360-degrees). Following administration of a therapy and/or an analgesic for the sprained ankle, another assessment of the subject is conducted in which the subject performs the same specific motions. If, for instance, the subject's motion measurements in terms of the body's ability to maintain stability and coordination has improved by 50 percent, a medical professional can make the determination that the subject's condition has improved 50 percent over an initial baseline in a specific timeframe after a treatment has been given (e.g., four hours after administration of 800 mg of ibuprofen). Such an assessment can also be used to maximize dosing of a patient for efficacy while also minimizing the potential side effects of over-dosing.

Moreover, a significant number of people possess a specific genetic variation in their metabolic enzymes which significantly reduces the effect of opioid analgesics and can increase their chances of becoming addicted to opioids. Testing for such genetic variation is expensive and time consuming and is not a prerequisite to having opioids prescribed by care givers. The present invention is capable of being used to non-invasively assess whether opioids are effective for a particular patient by testing for opioid insensitivity and for the presence of the aforementioned mutation. The present system can help clinicians efficiently determine whether an opioid is effective for its intended therapeutic effect and, if not, look for alternative therapies, reducing the risk of addiction and overdose. Thus, using the present system accommodates prevention of extra doses of a drug being given beyond the therapeutic value of that drug.

The present system is further capable of use for motion assessment of patients before orthopedic surgeries, such as but not limited to knee replacement, hip replacement, anterior cruciate ligament (ACL) reconstruction, posterior cruciate ligament (PCL) reconstruction, ulnar collateral ligament (UCL) reconstruction, or torn menisci repair, and to track patient recovery from surgery. Patients can be assessed using the present system prior to surgery and on one or more occasions after surgery and during recovery. The present invention is capable of being used to make objective and quantifiable measurements relating to the therapeutic effect of the procedure by comparing measurements from before and after surgery. Furthermore, the present invention is capable of objectively tracking the progress and/or rate of recovery.

In further exemplary embodiments of the present invention, patients can be sent home with appropriate technology and devices to perform automated assessments at home and transmit such assessments to medical professionals for review. The present invention is further capable of being configured for use in physical therapy and rehabilitation to assess and track subjects' conditions and to quantify and better understand therapeutic outcomes.

In additional embodiments, the present system is capable of being used to assess subjects and make informed predictions as to their susceptibility to particular musculoskeletal injuries. Moreover, the present invention can be used identify musculoskeletal or central nervous system (CNS) pathologies at an earlier stage in their development than traditional subjective human assessment.

In an exemplary embodiment of the present invention, the system is capable of use in association with a test for detecting intoxication. In such an embodiment, an IMU device is positioned on the body of a subject, and mathematical models created to differentiate normal from impaired individuals based on prescribed movements and/or actions, such as but not limited to walking in a straight line or standing still in an upright posture with eyes closed, are used to analyze motion data.

In an exemplary embodiment, the present system is utilized as a part of a field sobriety test. In this embodiment, an IMU sensor is attached to an individual suspected of driving while impaired, and the present system is used to process and analyze the data in real time or near real time. Data collected using on-body, multi-axial sensors have shown normal body movements (e.g., walking in a straight line) are generalizable across age, sex, and body type. In this embodiment, the operator of a motor vehicle believed to be impaired is evaluated in a three-dimensional space, which has the potential to detect abnormalities with more sensitivity.

In this embodiment, results are provided to a law enforcement officer in the field, and if the system measurements show the subject is potentially impaired, the subject is apprehended or the officer can further evaluate the subject with more invasive testing (e.g., blood and urine samples). In an embodiment, if the system measurements in the field do not show signs of impairment, the officer can release the subject after issuing any traffic citations, etc. as necessary.

In an exemplary embodiment, an IMU of the present invention utilized for a field sobriety test is an edge device having a self-contained microprocessor configured for processing acceleration and rotation data. In a preferred embodiment, the IMU sensor further includes an indicator light, such as but not limited to an LED light, configured for lighting up green, yellow, or red, depending on the data processed by the system. In such an embodiment, the system is configured to light up green if no indication of impairment is detected, red if probable or definite impairment is detected, and yellow if the data is inconclusive. Alternatively, an IMU of the present invention can include an indicator light which only illuminates in one color and can be programmed to illuminate or not illuminate in a certain sequence depending on the analyzed data from the IMU. In further embodiments, the IMU device is configured to illuminate an indicator light in one particular color if impairment is detected and illuminate in a different color if impairment is not detected or inconclusive.

In an embodiment, the law enforcement officer's body camera and/or vehicle camera are utilized in conjunction with the IMU for computer vision features of the present system. In other embodiments, a separate system camera and associated stand is set up in the field for computer vision features of the present system.

In an exemplary embodiment, the present system is configured to measure idiotypic behaviors of a subject and to quantify such behaviors to understand what particular intoxicant is present and at what level. For example, the effect of intoxication with tetrahydrocannabinol (THC), the main psychoactive compound in cannabis, and its action at the body's endocannabinoid system results in a distinct neuromuscular response that differs from other intoxicants. Such interaction can be observed and measured using the present system, which results in a mathematical or algorithmic description of THC intoxication by measurement and quantification of “behavior” based on a set of defined assessments of the intoxicated individual.

In additional embodiments of the present invention, the system is configured with algorithms for measurement and detection of a variety of neuroactive substances, particularly substances of abuse. The effects of various neuroactive drugs on specific areas of the brain and nervous system produce idiotypic behaviors which are dose related. The present system allows for identification of such behaviors as a function of a specific drug and its dose.

In a further exemplary embodiment, the present system is utilized for the objective and quantitative assessment of opioid efficacy. Opioid insensitivity in patients is a significant problem and is related to both misuse and abuse of opioids. Accordingly, more precise and personalized approaches to opioid prescription is needed in medicine, such as testing people for their level of sensitivity to prescribed opioids. Typically, that testing involves invasive and expensive approaches. In contrast, embodiments of the present invention allow for non-invasive and rapid assessment of opioid efficacy in patients related to pain levels the patients are experiencing, especially in orthopedic situations. Further, the present invention can help to distinguish between “real” pain due to a diagnosed pathology and a patient claiming to be in pain when not actually in pain simply to get more opioids. Thus, use of the present invention can improve assessments of patients receiving opioid therapy for effectiveness while also supporting management of opioid medications, thus addressing the problems of addiction and overdose, drug efficacy, safety, and overall cost of treatment.

Additionally, the present invention is capable of application to any situation where an impaired individual might impact the safety of others in a deleterious fashion. For example, the present invention can be extended to include pairwise comparisons of individuals using baseline data collected as part of standard business practices. Randomized follow-up screening tests of these individuals gathers data for comparison to the individuals' baseline measurements. Such screening tests could assess an individual's ability to perform critical job functions (e.g., surgical procedures, operation of machinery) that are highly dependent upon the individual's motor coordination.

In additional embodiments, the present system is capable of being used to assess subjects and make informed predictions related to physical performance. Specifically, the present system can evaluate, quantify, and monitor physical performance assessments related to exercise, conditioning, training, and competitive athletic activity. Physical performance functions can be recorded and analyzed by the system to provide measurable data related to improved or degraded performance of specific physical motions associated with exercise, conditioning, training, and competitive athletic activity. Such a system can be used to monitor and optimize athletic performance, to evaluate possible behaviors that may increase the level of performance or identify behaviors that may increase the risk of injury. Further, the system can be used by individuals to create a digital twins of their physical capabilities related to the musculoskeletal systems, and such digital twins can be used to track and measure their physical performance and identify limitations or improvements specific to the individual as a function of a conditioning or exercise regimen. In embodiments, the present system and method for quantifying bodily motion is utilized for tracking physical strength training and/or weight loss over time.

In additional embodiments of the present invention, the system further utilizes three-dimensional (3D) imaging via infrared or LIDAR. Such 3D imaging in embodiments of the present system expands camera data from (x,y) coordinates in time to (x,y,z) coordinates in time. Such embodiments provide greater image resolution and allow for the application of augmented reality (AR) and/or virtual reality (VR) technology to aid assessment. In an embodiment of the present invention utilizing real time or near real time 3D imaging, a 3D image of a patient or subject can be overlaid with output parameters on appropriate parts of the patient or subject's body indicating deviations from either baseline condition or a global nominal model. In another embodiment, a real time or near real time VR view of a patient or subject is utilized. For instance, a clinician can wear VR glasses or goggles while a patient performs standardized movements, and the clinician is informed of an assessment of those movements quantitatively in real time or near real time. In another embodiment, a law enforcement officer or medical professional can wear VR glasses or goggles while a subject performs standardized movements, and the officer or medical professional is informed of an impairment assessment of those movements quantitatively in real time or near real time.

Certain terminology is used in the description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology shall include the words specifically mentioned, derivatives thereof, and words of similar meaning.

As used in this specification and the appended claims, the singular forms “a”, “an”, and “the” include plural references unless the context clearly dictates otherwise. Thus, for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means ±5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder. “Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

It is also to be understood that the following claims are intended to cover all of the generic and specific features of the invention herein described, and all statements of the scope of the invention which, as a matter of language, might be said to fall therebetween.

Having thus described the invention, what is claimed as new and desired to be secured by Letters Patent is:

1. A system for quantifying bodily motion for diagnostics, progress tracking, and treatment, the system comprising:
a multi-axis inertial measurement device (IMU) selectively attachable to a subject, said IMU comprising an accelerometer and a gyroscope and configured for detecting acceleration and rotation data of said subject;
a system computing device in communication with said IMU and having a microprocessor;
said microprocessor configured to process acceleration and rotation data of said subject through a series of standardized movements to create a digital representation of bodily motion of said subject through space and time;
a user interface in communication with said system computing device and viewable on a display; and
said user interface configured for displaying said digital representation of bodily motion of said subject synchronized with time of completion of said series of standardized movements.

2. The system of claim 1, further comprising:
a camera in communication with said system computing device and configured for recording video of said subject through said series of standardized movements;
wherein said microprocessor is configured for processing video from said camera and identifying translation of body parts of said subject through space and time.

3. The system of claim 2, wherein:
said microprocessor processing video from said camera comprises identifying key points on said subject, each key point associated with an anatomical landmark of said subject.

4. The system of claim 3, further comprising:
a data synchronization module configured for synchronizing acceleration, rotation, key point coordinate position in space and time.

5. The system of claim 1, further comprising:
a database with memory in communication with said system computing device;
wherein said database is configured for storing historical bodily motion data for said subject.

6. The system of claim 5, wherein:
said database is further configured for storing global nominal model motion data.

7. The system of claim 1, further comprising:
a data gateway and a message broker in communication with said system computing device and said IMU.

8. The system of claim 1, wherein:
said IMU comprises a 9-axis absolute orientation sensor combining a 3-axis solid-state acceleration sensor, a 3-axis gyroscope, and a 3-axis geomagnetic sensor.

9. The system of claim 1, wherein:
said IMU sensor captures data at 30 to 100 Hz.

10. The system of claim 1, wherein:
said system computing device is self-contained within said IMU.

11. The system of claim 10, wherein:
said IMU further comprises an indicator light configured for illuminating when said digital representation of bodily motion of said subject shows a predetermined characteristic.

12. A method of detecting and treating a medical condition of a patient using a system for quantifying bodily motion, the method comprising:
placing a multi-axis inertial measurement device (IMU) on said patient;
pointing a camera in position to record bodily motion of said patient;
said patient performing a series of standardized movements;
said IMU detecting acceleration and rotation data of said patient through said series of standardized movements;
said camera recording video of said patient through said series of standardized movements;
providing a system computing device having a microprocessor configured to process said acceleration and rotation data of said patient and to process video from said camera and identify translation of body parts of said patient through space and time;
said system computing device creating a baseline digital representation of bodily motion of said patient through space and time; and
a user interface displaying said baseline digital representation of bodily motion of said patient.

13. The method of claim 12, wherein said system for quantifying bodily motion further comprises database with memory in communication with said system computing device, the method further comprising the step of:
said database storing said baseline digital representation of bodily motion of said patient.

14. The method of claim 13, further comprising the steps of:
applying a treatment to said medical condition;
said patient performing a second repetition of said series of standardized movements;
said IMU detecting acceleration and rotation data of said patient through said second repetition of said series of standardized movements;
said camera recording video of said patient through said second repetition of said series of standardized movements;
said system computing device creating an active digital representation of bodily motion of said patient through space and time; and
comparing said active digital representation of bodily motion of said patient to said baseline digital representation of bodily movement of said patient to determine efficacy of said treatment.

15. The method of claim 14, further comprising the steps of:
said database storing historical digital representation of bodily motion data; and
comparing said active digital representation of bodily motion of said patient to said historical digital representation of bodily motion data to determine efficacy of said treatment.

16. The method of claim 14, further comprising the steps of:
said database storing global nominal model motion data; and
comparing said active digital representation of bodily motion of said patient to said global nominal model motion data.

17. A method of detecting impairment of a subject using a system for quantifying bodily motion, the method comprising:
placing a multi-axis inertial measurement device (IMU) on said subject;
said subject performing a series of standardized movements;
said IMU detecting acceleration and rotation data of said subject through said series of standardized movements;
providing a microprocessor configured to process said acceleration and rotation data of said subject;
said microprocessor creating an active digital representation of bodily motion of said subject through space and time;
said microprocessor determining if said active digital representation of bodily motion of said subject shows a characteristic of impairment.

18. The method of claim 17, wherein:
said microprocessor determining if said active digital representation of bodily motion of said subject shows a characteristic of impairment comprises comparing said active digital representation of bodily motion of said subject to a baseline digital representation of bodily motion of said subject.

19. The method of claim 17, wherein:
said microprocessor determining if said active digital representation of bodily motion of said subject shows a characteristic of impairment comprises comparing said active digital representation of bodily motion of said subject to a global nominal motion model.

20. The method of claim 17, further comprising the steps of:
pointing a camera in position to record bodily motion of said subject;
said camera recording video of said subject through said series of standardized movements;
wherein said microprocessor is configured to process video from said camera and identify translation of body parts of said subject through space and time.
